(19) United States
(12) Patent Application Publication
(10) Pub. No.: US 2019/0330185 A1
(43) Pub. Date: Oct. 31, 2019

(54) 4-((6-(2-(2,4-DIFLUOROPHENYL)-1,1-DIFLUORO-2-HYDROXY-3-(1H-1,2,4-TRIAZOL-1-YL)PROPYL)PYRIDIN-3-YL)OXY)BENZONITRILE AND PROCESSES OF PREPARATION

(71) Applicant: DOW AGROSCIENCES LLC, Indianapolis (IN)

(72) Inventors: Qiang Yang, Zionsville, IN (US); Yan Hao, Zionsville, IN (US); Sarah Ryan, Indianapolis, IN (US)

(21) Appl. No.: 16/507,443

(22) Filed: Jul. 10, 2019

Related U.S. Application Data
(63) Continuation of application No. 15/776,615, filed on May 16, 2018, now abandoned.
(60) Provisional application No. 62/256,548, filed on Nov. 17, 2015.

(51) Int. Cl. C07D 401/06

(52) U.S. Cl. CPC C07D 401/06

(57) ABSTRACT

Provided herein is a process for the preparation of 4-((6-(2-(2,4-difluorophenyl)-1,1-difluoro-2-hydroxy-3-(1H-1,2,4-triazol-1-yl-propyl)pyridin-3-yl)oxy)benzonitrile.

### CROSS-REFERENCE TO RELATED APPLICATIONS

This application claims priority to U.S. Provisional Application No. 62/256,548, filed Nov. 17, 2015, which is incorporated herein by reference in its entirety.

### FIELD

Provided herein is 4-((6-(2-(2,4-difluorophenyl)-1,1-difluoro-2-hydroxy-3-(1H-1,2,4-triazol-1-yl)propyl)pyridin-3-yl)oxy)benzonitrile and processes of preparation.

### BACKGROUND

U.S. patent application Ser. Nos. 13/527,387, 13/527,426 and 13/528,283 describe inter alia certain metalloenzyme inhibitor compounds and their use as fungicides. The disclosure of each application is expressly incorporated by reference herein. Each of these patent applications describe various routes to generate metalloenzyme inhibiting fungicides. It may be advantageous to provide more direct and efficient methods for the preparation of metalloenzyme inhibiting fungicides and related compounds, e.g., by the use of reagents and/or chemical intermediates which provide improved time and cost efficiency.

### SUMMARY OF THE DISCLOSURE

Provided herein is the compound 4-((6-(2-(2,4-difluorophenyl)-1,1-difluoro-2-hydroxy-3-(1H-1,2,4-triazol-1-yl)propyl)pyridin-3-yl)oxy)benzonitrile (I) and processes for its preparation. In one embodiment, provided herein, is a process for the preparation of the compound of the Formula I:

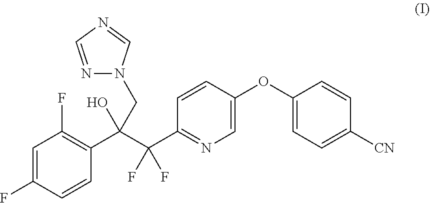 [SMILES: N#Cc1ccc(Oc2ccc(C(F)(F)C(O)(Cn3cncn3)c3ccc(F)cc3F)nc2)cc1]

which comprises contacting a compound of Formula II with a trialkylsulfoxonium halide, a base, and 1H-1,2,4-triazole.

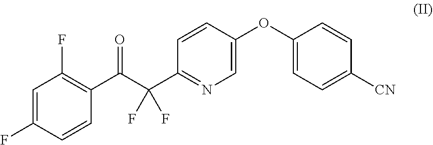 [SMILES: N#Cc1ccc(Oc2ccc(C(F)(F)C(=O)c3ccc(F)cc3F)nc2)cc1]

The term “halogen” or “halo” refers to one or more halogen atoms, defined as F, Cl, Br, and I. The term “hydroxyl” refers to an —OII substituent.

The term “organometallic” refers to an organic compound containing a metal, especially a compound in which a metal atom is bonded directly to a carbon atom.

Room temperature (RT) is defined herein as about 20° C. to about 25° C.

Throughout the disclosure, references to the compounds of Formula I are read as also including optical isomers and salts. Specifically, when compounds of Formula I contain a chiral carbon, it is understood that such compounds include optical isomers and racemates thereof. Exemplary salts may include: hydrochloride, hydrobromide, hydroiodide, and the like.

Certain compounds disclosed in this document can exist as one or more isomers. It will be appreciated by those skilled in the art that one isomer may be more active than the others. The structures disclosed in the present disclosure are drawn in only one geometric form for clarity, but are intended to represent all geometric and tautomeric forms of the molecule.

The embodiments described above are intended merely to be exemplary, and those skilled in the art will recognize, or will be able to ascertain using no more than routine experimentation, numerous equivalents of specific processes, materials and procedures. All such equivalents are considered to be within the scope of the invention and are encompassed by the appended claims.

### DETAILED DESCRIPTION

4-((6-(2-(2,4-difluorophenyl)-1,1-difluoro-2-hydroxy-3-(1H-1,2,4-triazol-1-yl)propyl)pyridin-3-yl)oxy)benzonitrile (I) is provided herein and may be prepared from 4-((6-(2-(2,4-difluorophenyl)-1,1-difluoro-2-oxoethyl)pyridin-3-yl)oxy)benzonitrile (II) as shown in Example 1.

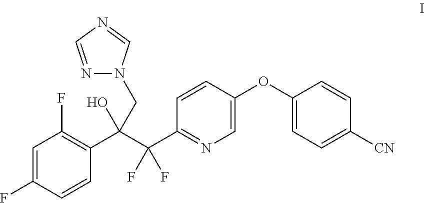 [SMILES: N#Cc1ccc(Oc2ccc(C(F)(F)C(O)(Cn3cncn3)c3ccc(F)cc3F)nc2)cc1]

### EXAMPLE 1

### Preparation of 4-((6-(2-(2,4-difluorophenyl)-1,1-difluoro-2-hydroxy-3-(1H-1,2,4-triazol-1-yl)propyl)pyridin-3-yl)oxy)benzonitrile (I)

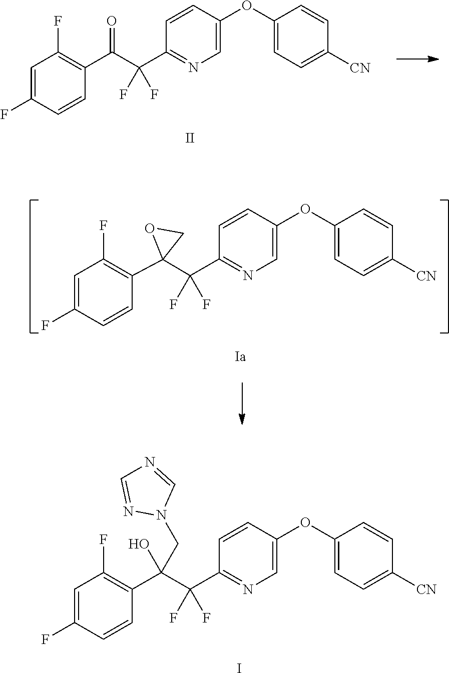

Method A: Potassium carbonate (32.6 g, 236 mmol) was charged to a suspension of trimethylsulfoxonium iodide (26.5 g, 118 mmol) in NMP (190 mL) at less than 5° C., and the reaction was stirred at 20° C. for 2 h to give a white suspension. 4-((6-(2-(2,4-Difluorophenyl)-1,1-difluoro-2-oxoethyl)pyridin-3-yl)oxy)benzonitrile (II) (38 g, 94 mmol) was added in one portion, and the reaction was stirred at 35° C. under N₂ for 18 h, at which point HPLC analysis indicated that the starting material was fully converted to the epoxide intermediate (Ia). 1H-1,2,4-Triazole (8.56 g, 123 mmol) was added, and the reaction was stirred at 60° C. for 18 h, at which point HPLC analysis showed about 10% epoxide intermediate (Ia) remaining. The reaction was further stirred at 80° C. for 1 h, at which point, HPLC analysis indicated that the reaction was complete. The mixture was allowed to cool to 20° C. and was poured into ice water (1200 mL). The resulting suspension was filtered, and the solid was dissolved in DCM (1200 mL). The solution was washed with brine (2×300 mL) and the organic layer was concentrated to about 200 mL. The resulting solution was purified by column chromatography (750 g silica) using EtOAc/hexanes as eluent to afford the desire product as a light yellow foam (39.2 g, 85% yield). ¹H NMR (400 MHz, CDCl₃) δ 8.36 (d, J=2.7 Hz, 1H), 8.15 (d, J=1.0 Hz, 1H), 7.74 (s, 1H), 7.73-7.67 (m, 2H), 7.58 (dd, J=8.7, 0.6 Hz, 1H), 7.51-7.44 (m, 1H), 7.42 (dd, J=8.7, 2.8 Hz, 1H), 7.15-7.03 (m, 2H), 6.81-6.68 (m, 2H), 6.27 (s, 1H), 5.40 (d, J=14.4 Hz, 1H), 4.93-4.82 (m, 1H); ESIMS m/z 470.0 ([M+H]⁺).

Method B: To a 100-mL, 3-neck, round bottom flask was charged trimethylsulfoxonium iodide (0.356 g, 1.618 mmol) and NMP (5 mL). NaOt-Bu (0.143 g, 1.488 mmol) was added at less than 25° C., and the reaction was stirred at 20° C. for 1 h. The reaction was cooled to less than −15° C. and 4-((6-(2-(2,4-difluorophenyl)-1,1-difluoro-2-oxoethyl)pyridin-3-yl)oxy)benzonitrile (II) (0.5 g, 1.294 mmol) was added. The reaction was stirred at less than −10° C. for 1 h, after which time HPLC analysis indicated that the starting material had been fully converted to the epoxide intermediate (Ia). 1H-1,2,4-Triazole (0.103 g, 1.488 mmol) and NaOt-Bu (0.143 g, 1.488 mmol) were added, and the reaction was heated at 40° C. for 6 h. The reaction was cooled to 20° C. and added with water (20 mL). The mixture was extracted with EtOAc (2×20 mL). The organics were concentrated to dryness and purified by column chromatography (40 g silica, 0-60% EtOAc/hexanes over 5 column volumes, hold for 5 volumes). Fractions containing pure product were concentrated to afford a colorless oil (400 mg, 66% yield). Analytical data were consistent with that of previously obtained samples.

Method C: To a 100-mL, 3-neck, round bottom flask was charged trimethylsulfoxonium bromide (0.560 g, 3.24 mmol) and NMP (5 mL). K₂CO₃ (1.073 g, 7.77 mmol) was added at less than 25° C., and the reaction was stirred at 20° C. for 1 h. 4-((6-(2-(2,4-Difluorophenyl)-1,1-difluoro-2-oxoethyl)pyridin-3-yl)oxy)benzonitrile (II) (1.0 g, 2.59 mmol) was added, and the reaction was stirred at 20° C. for 18 h, after which time HPLC analysis indicated that the reaction was incomplete. It was further stirred at 35° C. for 4 h, after which time HPLC analysis indicated that the starting material was consumed. 1H-1,2,4-Triazole (0.215, 3.11 mmol) was added, and the reaction was stirred at 20° C. for 18 h, at which point HPLC analysis indicated that the reaction was incomplete. It was further heated at 35° C. for 4 h, and cooled to 20° C. Water (20 mL) was added, and the reaction mixture was stirred for 30 min to afford a gummy precipitate, which was isolated by decanting off solvent. The crude product was purified by column chromatography (40 g silica, 0-50% EtOAc/hexanes over 10 min, hold for 15 min). Fractions containing pure product were concentrated to afford a white foam (0.89 g, 73% yield). Analytical data were consistent with that of previously obtained samples.

Method D: A 100-mL, 3-neck, round bottom flask was charged with trimethylsulfoxonium chloride (0.832 g, 6.48 mmol) and NMP (10 mL). K₂CO₃ (2.146 g, 15.554 mmol) was added at less than 25° C., and the reaction was stirred at 20° C. for 1 h. 4-((6-(2-(2,4-Difluorophenyl)-1,1-difluoro-2-oxoethyl)pyridin-3-yl)oxy)benzonitrile (II) (2.0 g, 5.18 mmol) was added, and the reaction was stirred at 20° C. for 18 h, after which time HPLC analysis indicated that the starting material was fully consumed. 1H-1,2,4-Triazole (0.43 g, 6.11 mmol) was added, and the reaction was stirred at 20° C. for 18 h, at which point HPLC analysis indicated that the reaction was complete. Water (25 mL) was added, and the reaction mixture was stirred for 30 min to afford a gummy precipitate, which was isolated by decanting off solvent. The crude product was purified by column chromatography (80 g silica, 0-50% EtOAc/hexanes over 10 min, hold for 15 min). Fractions containing pure product were concentrated to afford a white foam (1.5 g, 62% yield). Analytical data were consistent with that of previously obtained samples.

Method E: To a 250 mL jacketed reactor with the jacket set at 25° C. were added trimethylsulfoxonium bromide (6.16 g, 35.6 mmol), potassium carbonate (11.18 g, 81 mmol), and DMSO (37.5 mL). The slurry was stirred for 30 min then 4-((6-(2-(2,4-difluorophenyl)-1,1-difluoro-2-oxoethyl)pyridin-3-yl)oxy)benzonitrile (II) (12.5 g, 32.4 mmol) was added and the jacket was heated to 55° C. After 1 h, 1H-1,2,4-triazole (2.458 g, 35.6 mmol) was added and the mixture was stirred at 55° C. for 5 h. The jacket was turned down to 25° C. and 125 mL MTBE was added to the reaction then 125 mL water was added. The mixture was stirred vigorously for 30 min then was allowed to settle. The aqueous layer was removed and 125 mL water was added to the organic layer and the two were mixed for 15 min. 25 mL MTBE and 10 mL saturated brine were added and the layers mixed for 2 minutes then allowed to settle. The aqueous layer was removed from the reactor. The reactor was fitted with a distillation head and the jacket set to 65° C. 82 g of solvent were atmospherically distilled overhead (about 115 mL) then methanol (53 g, about 70 mL) was added. Distillation was continued until the overhead temperature was 65° C. and a total of 130 g of solvent had been distilled overhead (about 110 g MTBE and about 20 g MeOH; 33 g of methanol remained in the reactor). The jacket was cooled to 60° C. and water (3.4 g) was added dropwise. The mixture was then seeded with compound I. Additional water (3.2 g) was added slowly causing precipitation of more solids. The slurry was cooled to 20° C. over 4 h. After stirring at 20° C. for 1 h the solids were isolated by filtration and washing the reaction vessel with the mother liquor to clear out the solids. The solids were washed with 2:1 methanol/water w/w (2×10 mL). The solids were air dried to constant mass giving 4-((6-(2-(2,4-difluorophenyl)-1,1-difluoro-2-hydroxy-3-(1H-1,2,4-triazol-1-yl)propyl)pyridin-3-yl)oxy)benzonitrile (I) (10.08 g, 20.40 mmol, 63.0% yield) as a tan solid. Analytical data were consistent with that of previously obtained samples.

Method F: To a 250 mL jacketed reactor set at 25° C. were added trimethylsulfoxonium bromide (6.16 g, 35.6 mmol), potassium carbonate (11.18 g, 81 mmol), THF (62.6 mL), and water (12.51 mL). This slurry was stirred at 25° C. for 15 min and then 44(64242,4-difluorophenyl)-1,1-difluoro-2-oxoethyl)pyridin-3-yl)oxy)benzonitrile (TT) (12.5 g, 32.4 mmol) was added and the mixture was stirred at 60° C. overnight. The jacket was cooled to 25° C., water (37.5 mL) was added and the layers mixed for 5 min. The aqueous layer was removed from the reactor. The organic layer was distilled atmospherically with jacket at 85° C. After 40 mL was distilled overhead, 37.5 mL DMSO was added. Distillation was continued with only 5 mL more solvent coming overhead. The jacket was cooled to 55° C. leaving about 20 mL THF in the reaction mixture. Potassium carbonate (11.18 g, 81 mmol) followed by 1H-1,2,4-triazole (2.458 g, 35.6 mmol) were added. The reaction was stirred at 55° C. for 5 h then MTBE (125 mL) and water (125 mL) were added and mixed for 15 min. The layers were separated. The organic layer was washed with a mixture of 125 mL water and 20 mL brine. The organic layer left in the jacketed reactor was distilled atmospherically. After 67 g of solvent was distilled overhead, 55.7 g methanol was added and distillation continued until 47 g more solvent had come overhead. The dark brown solution was cooled to 60° C. and then 3.02 g water was added slowly and the mixture was seeded. An additional 8.5 g water was added giving about 3:1 methanol/water w/w. The mixture was cooled to 20° C. over 2 h and the slurry was held at 20° C. overnight. The solids that formed were isolated by filtration, washing the reactor with the mother liquor. The solids were washed with 3:1 methanol/water w/w (20 g) and air dried to constant mass giving 4-((6-(2-(2,4-difluorophenyl)-1,1-difluoro-2-hydroxy-3-(1H-1,2,4-triazol-1-yl)propyl)pyridin-3-yl)oxy)benzonitrile (I) (11.62 g, 24.76 mmol, 77% yield) as a tan solid. ¹H NMR (400 MHz, DMSO-d₆) δ 8.47 (d, J=2.7 Hz, 1H), 8.36 (s, 1H), 7.99-7.89 (m, 2H), 7.71 (s, 1H), 7.69 (dd, J=8.7, 2.8 Hz, 1H), 7.51 (d, J=8.7 Hz, 1H), 7.30-7.19 (m, 3H), 7.13 (ddd, J=12.0, 9.2, 2.6 Hz, 1H), 7.05 (s, 1H), 6.88 (td, J=8.5, 2.6 Hz, 1H), 5.35 (d, J=14.6 Hz, 1H), 4.83 (d, J=14.6 Hz, 1H). ¹⁹F NMR (376 MHz, DMSO-d₆) δ −102.83 (td, J=22.5, 21.9, 9.2 Hz), −107.66 (dd, J=21.7, 13.5 Hz), −110.46 (d, J=9.4 Hz). ESIMS m/z 470.2 [(M+H)⁺].

The processes described herein may be conducted at temperatures ranging from about −20° C. to about 100° C., or from about 20° C. to about 80° C.

Solvents that may be used in the processes described herein may include at least one of dimethylsulfoxide (DMSO), dimethylformamide (DMF), tetrahydrofuran (THF), sulfolane, water, and N-methyl-2-pyrrolidone (NMP).

Bases that may be used in the processes described herein may include metal carbonates such as, for example, potassium carbonate and sodium carbonate, metal alkoxides such as, for example, potassium tert-butoxide, or metal bicarbonates such as, for example, sodium and potassium bicarbonate.

What is claimed is:

1. A method of making a compound of Formula I 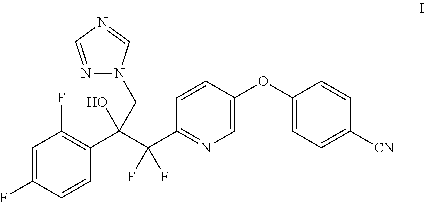 [SMILES: N#Cc1ccc(Oc2ccc(C(F)(F)C(O)(Cn3cncn3)c3ccc(F)cc3F)nc2)cc1]
comprising the step of contacting a compound of Formula II 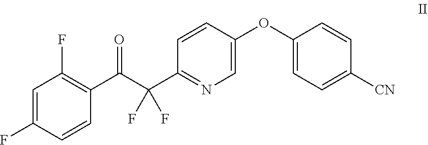 [SMILES: N#Cc1ccc(Oc2ccc(C(F)(F)C(=O)c3ccc(F)cc3F)nc2)cc1]
with a trialkylsulfoxonium halide, a base, and 1H-1,2,4-triazole.

2. The method of claim 1, wherein the trialkylsulfoxonium halide is one of trimethylsulfoxonium iodide, trimethylsulfoxonium bromide and trimethylsulfoxonium chloride.

3. The method of claim 1, wherein the base may be selected from the group including metal carbonates, metal alkoxides and metal bicarbonates.

4. The method of claim 1, wherein the base may be one of potassium carbonate and sodium tert-butoxide.

5. The method of claim 1 further comprising a solvent selected from the group including dimethylsulfoxide (DMSO), dimethylformamide (DMF), sulfolane, tetrahydrofuran (THF), water, N-methyl-2-pyrrolidone (NMP), and mixtures thereof.

6. The method of claim 1 further comprising a solvent selected from the group including THF, water, DMSO, and mixtures thereof.

7. The method of claim 1 wherein the contacting is carried out from about −20° C. to about 100° C.

8. The method of claim 1 wherein the contacting is carried out from about 20° C. to about 80° C.
